(19) United States
(12) Patent Application Publication
(10) Pub. No.: US 2022/0064706 A1
(43) Pub. Date: Mar. 3, 2022

(54) NUCLEIC ACID RELEASE AGENT, NUCLEIC ACID PCR AMPLIFICATION METHOD AND PCR AMPLIFICATION KIT

(71) Applicant: SANSURE BIOTECH INC., Changsha, Hunan (CN)

(72) Inventors: Lizhong DAI, Hunan (CN); Bozhi JI, Hunan (CN); Wenqu ZHANG, Hunan (CN); Zhongping DENG, Hunan (CN); Wenzhou FAN, Hunan (CN)

(73) Assignee: SANSURE BIOTECH INC., Hunan, Hunan (CN)

(21) Appl. No.: 17/421,390

(22) PCT Filed: Apr. 29, 2019
(86) PCT No.: PCT/CN2019/085066

§ 371 Date: Jul. 8, 2021

(30) Foreign Application Priority Data
Jan. 8, 2019 (CN) 201910014324.3

(51) Int. Cl. C12Q 1/686; C12Q 1/6848; C12N 9/58

(52) U.S. Cl. CPC C12Q 1/686; C12Y 304/21014; C12N 9/58; C12Q 1/6848

(57) ABSTRACT

A nucleic acid release agent, a PCR amplification method and a PCR amplification kit are provided. The nucleic acid release agent includes Tris-HCl, sodium chloride, potassium chloride, tween 20, Triton X-100, ethyl phenyl polyethylene glycol and a strong base; wherein the molar concentration of Tris-HCl is 0.5 mM to 500 mM, the molar concentration of sodium chloride is 20 mM to 500 mM, the volume percentage of Tween 20 is 0.1% to 2%, the volume percentage of Triton X-100 is 0.1% to 3%, the volume percentage of ethyl phenyl polyethylene glycol is 0.1% to 3%, the mass concentration of potassium chloride is 5 mg/mL to 8 mg/mL and the mass concentration of the strong base is 2 mg/mL to 50 mg/mL.

### BACKGROUND

### Technical Field

The present disclosure relates to the field of molecular biology, in particular to a nucleic acid release agent, a method for PCR amplification of a nucleic acid and a PCR amplification kit.

### Description of Related Art

Polymerase chain reaction (PCR) is a molecular biological technique for amplifying a specific nucleic acid fragment, importantly characterized by massive enrichment and increase of a trace amount of nucleic acid, so as to facilitate the detection of the trace amount of nucleic acid. The commonly used PCR method in medical diagnosis is mainly a real-time fluorescent quantitative PCR (qPCR) method based on dual fluorescent probes. Targets for in vitro diagnosis using the qPCR method mainly include human genomic DNA, DNA viruses, bacteria, fungi, RNA viruses, and the like. However, since RNA has a single-stranded structure, which is unstable and easily degraded, a process of sample treatment is subjected to very stringent requirements, and a more complicated method is required for pretreatment, nucleic acid extraction and purification of the RNA sample to be amplified to obtain the pure nucleic acid, then the detection can be performed to obtain a stable result.

### SUMMARY

Accordingly, it is necessary to provide a nucleic acid release agent that can simplify the PCR amplification method of RNA samples.

The present disclosure provides a nucleic acid release agent including Tris-HCl, sodium chloride, potassium chloride, Tween 20, Triton X-100, ethyl phenyl polyethylene glycol and a strong base; wherein Tris-HCl has a molar concentration ranging from 0.5 mM to 500 mM, sodium chloride has a molar concentration from 20 mM to 500 mM, potassium chloride has a mass concentration ranging from 5 mg/mL to 8 mg/mL, Tween 20 has a volume percentage ranging from 0.1% to 2%, Triton X-100 has a volume percentage ranging from 0.1% to 3%, ethyl phenyl polyethylene glycol has a volume percentage ranging from 0.1% to 3%, and the strong base has a mass concentration ranging from 2 mg/mL to 50 mg/mL.

In the nucleic acid release agent of the present disclosure, a certain proportion of the strong base lyses cells, which release nucleic acids. Sodium chloride and potassium chloride protect nucleic acids by coordinating the balance of intracellular and extracellular ions. Tris-HCl is for keeping a pH value stable during cell lysis and better compatible with a PCR reaction solution in subsequent amplification. Triton X-100, on the one hand, can protect nucleic acids, especially single-stranded RNA, allowing RNA to be stored in an alkaline environment. On the other hand, Triton X-100 with potassium chloride in a certain ratio can reduce the inhibitory effect of the strong alkaline environment on the enzyme in the PCR reaction, thereby ensuring the amplification efficiency of RNA. Tween 20 and ethyl phenyl polyethylene glycol can protect reverse transcriptase, so that the reverse transcriptase works normally in the alkaline environment. Through the synergy of the above components, the nucleic acid release agent of the present disclosure enables a sample containing RNA to directly release RNA at room temperature, thereby avoiding the problem of sample contamination caused by aerosol generated by heating, and effectively preventing RNA from degrading in the alkaline environment. More importantly, it can be directly mixed with the PCR reaction solution for PCR amplification to complete the amplification without the need for complicated nucleic acid extraction and purification processes. This truly realizes a one-chamber, pollution-free, simple and rapid RNA amplification and detection, with high detection sensitivity and good repeatability.

In one of the embodiments, the nucleic acid release agent further includes betaine and bovine serum albumin, wherein betaine has a mass concentration ranging from 0.1 mg/mL to 20 mg/mL, and bovine serum albumin has a mass concentration ranging from 5 mg/mL to 100 mg/mL.

In one of the embodiments, the nucleic acid release agent further includes proteinase K and lithium dodecyl sulfate, wherein proteinase K has a mass concentration ranging from 0.02 mg/mL to 1.5 mg/mL, and lithium dodecyl sulfate has a mass concentration ranging from 0.4 mg/mL to 30 mg/mL.

The present disclosure also provides a method for PCR amplification of a nucleic acid, including steps of: mixing the nucleic acid release agent as described above with a sample, placing the mixture at 25° C. to 60° C. for 2 min to 10 min, and adding a PCR reaction solution for PCR amplification.

In one of the embodiments, the method further includes a sample pretreatment step of mixing the sample with polyethylene glycol followed by centrifugation to collect a precipitate before mixing the nucleic acid release agent with the sample.

In one of the embodiments, the PCR amplification of intestinal viruses in the sample is performed at following conditions:
reverse transcription at 48° C. to 52° C. for 28 min to 32 min;
thermal denaturation at 93° C. to 97° C. for 0.9 min to 1.1 min;
several cycles of amplification at 93° C. to 97° C. for 13 sec to 17 sec followed by 53° C. to 57° C. for 28 sec to 32 sec;
cooling at 23° C. to 27° C. for 8 sec to 12 sec.

In one of the embodiments, the PCR amplification of hepatitis C virus in the sample is performed at following conditions:
pre-denaturation and enzyme activation at 93° C. to 97° C. for 0.9 min to 1.1 min;
reverse transcription at 58° C. to 62° C. for 28 min to 32 min;
thermal denaturation at 93° C. to 97° C. for 0.9 min to 1.1 min;
several cycles of amplification at 93° C. to 97° C. for 13 sec to 17 sec followed by 58° C. to 62° C. for 28 sec to 32 sec;
cooling at 23° C. to 27° C. for 8 sec to 12 sec.

In one of the embodiments, the PCR amplification of respiratory viruses in the sample is performed at following conditions:
reverse transcription at 48° C. to 52° C. for 28 min to 32 min;
thermal denaturation at 93° C. to 97° C. for 0.9 min to 1.1 min;
several cycles of amplification at 93° C. to 97° C. for 13 sec to 17 sec followed by 58° C. to 62° C. for 28 sec to 32 sec;
cooling at 23° C. to 27° C. for 8 sec to 12 sec.

In one of the embodiments, the PCR amplification of respiratory bacteria in the sample is performed at following conditions:
UDG enzyme reaction at 48° C. to 52° C. for 1.9 min to 2.1 min;
thermal denaturation at 92° C. to 96° C. for 2.9 min to 3.1 min;
several cycles of amplification at 92° C. to 96° C. for 8 sec to 12 sec followed by 58° C. to 62° C. for 18 sec to 22 sec;
extension and fluorescence collection at 73° C. to 77° C. for 18 sec to 22 sec; melting curve: 62° C. to 75° C.

The present disclosure also provides a PCR amplification kit, including the nucleic acid release agent as described above and a PCR reaction solution.

### BRIEF DESCRIPTION OF THE DRAWINGS

FIG. 1 shows real-time fluorescence quantitative PCR amplification curves in Examples 1-25.

FIG. 2 shows real-time fluorescence quantitative PCR amplification curves in Examples 26-50.

FIG. 3 shows real-time fluorescence quantitative PCR amplification curves in Examples 51-70.

FIG. 4 shows real-time fluorescence quantitative PCR amplification curves in Examples 71-90.

### DESCRIPTION OF THE EMBODIMENTS

To make the present disclosure easy to understand, a more comprehensive description of the present disclosure will be given below, and better embodiments of the present disclosure are given below. However, the present disclosure can be implemented in many different forms and shall not be limited to the embodiments described herein. On the contrary, the purpose of providing these embodiments is to provide a more thorough and comprehensive understanding of the disclosure of the present disclosure.

Unless otherwise defined, all technical and scientific terms used herein have the same meanings as those generally understood by those skilled in the art of the present disclosure. The terms used in the specification of the present disclosure are only for the purpose of describing specific embodiments, rather than limiting the present disclosure. The term “and/or” as used herein includes any and all combinations of one or more related listed items.

In an embodiment of the present disclosure, a nucleic acid release agent includes Tris-HCl, sodium chloride, potassium chloride, Tween 20, Triton X-100, ethyl phenyl polyethylene glycol and a strong base; wherein Tris-HCl has a molar concentration ranging from 0.5 mM to 500 mM, sodium chloride has a molar concentration from 20 mM to 500 mM, potassium chloride has a mass concentration ranging from 5 mg/mL to 8 mg/mL, Tween 20 has a volume percentage ranging from 0.1% to 2%, Triton X-100 has a volume percentage ranging from 0.1% to 3%, ethyl phenyl polyethylene glycol has a volume percentage ranging from 0.1% to 3%, and the strong base has a mass concentration ranging from 2 mg/mL to 50 mg/mL.

In order to achieve the effect of lysis, a lysis solution needs to be alkaline, but RNA is easily degraded in an alkaline environment. Moreover, a reverse transcriptase, which is required for PCR amplification and detection of RNA, is relatively fragile compared with the thermally stable Taq DNA polymerase and is easily inhibited in an alkaline environment, resulting in an inability to achieve the effect of reverse transcription. Therefore, RNA in the lysis solution generally cannot be directly amplified and detected by PCR. At present, pre-treating methods for PCR amplification and detection of RNA samples mainly include a boiling lysis method and a magnetic bead method. In the boiling lysis method, nucleic acids in a sample are released by boiling under the action of a lysis buffer and dissolved in the lysis buffer. While the cells are lysed in a boiling water bath, the proteins and chromosomes of the cells are denatured. Then the denatured proteins and other impurities are removed by centrifugation, and the nucleic acids in the supernatant are recovered for PCR amplification. However, coagulation of the proteins subjected to boiling at a high temperature causes part of the nucleic acids to be wrapped and run off with the centrifugation, leading directly to a decrease in the amount of template nucleic acids in the supernatant, which reduces the sensitivity of subsequent amplification and detection. In addition, aerosols are easily produced by boiling and heating, leading to sample contamination, which will result in false-positive results in subsequent detection. In the magnetic bead method, cells are lysed in a lysis solution, and free nucleic acid molecules are specifically adsorbed to the surface of the magnetic particles, while impurities such as proteins are not adsorbed but remained in the solution. After reacting for a certain period of time, the magnetic particles are separated from the liquid under the action of a magnetic field, followed by elution with an eluent to obtain pure nucleic acids. However, the magnetic bead method has complicated operation, requires a sample size that is generally 200 to 600 microliters, and has a very high demand on equipments, giving limitations on its promotion and use.

Based on the above mechanism, in the nucleic acid release agent of this embodiment, a certain proportion of the strong base lyses cells, which release nucleic acids.. Sodium chloride and potassium chloride protect nucleic acids by coordinating the balance of intracellular and extracellular ions. Tris-HCl is for keeping a pH value stable during cell lysis and is better compatible with a PCR reaction solution in subsequent amplification. Triton X-100, on the one hand, can protect nucleic acids, especially single-stranded RNA, allowing RNA to be stored in an alkaline environment. On the other hand, Triton X-100 with potassium chloride in a certain ratio can reduce the inhibitory effect of the strong alkaline environment on the enzyme in the PCR reaction, thereby ensuring the amplification efficiency of RNA. Tween 20 and ethyl phenyl polyethylene glycol can protect reverse transcriptase, so that the reverse transcriptase works normally in the alkaline environment. Through the synergy of the above components, the nucleic acid release agent of this embodiment enables a sample containing RNA to directly release RNA from at room temperature, thereby avoiding the problem of sample contamination caused by aerosol generated by heating and effectively preventing RNA from degrading in the alkaline environment. More importantly, it can be directly mixed with the PCR reaction solution for PCR amplification to complete the amplification, without the need for complicated nucleic acid extraction and purification processes. This truly realizes a one-chamber, pollution-free, simple and rapid RNA amplification and detection, with high detection sensitivity and good repeatability. It can be understood that the nucleic acid release agent can be used not only for PCR amplification and detection of RNA samples, but also for multiple and combined amplification and detection of DNA samples or mixed samples of RNA and DNA.

Specifically, the strong base is sodium hydroxide or potassium hydroxide, and the like, which can be selected as required.

In a specific example, the nucleic acid release agent further includes betaine and bovine serum albumin, wherein betaine has a mass concentration ranging from 0.1 mg/mL to 20 mg/mL, and bovine serum albumin has a mass concentration ranging from 5 mg/mL to 100 mg/mL. The certain proportion of betaine and bovine serum albumin added may collaborate with Triton X-100 to better protect RNA under alkaline conditions and prevent polymerase and reverse transcriptase from their denaturation, thereby guaranteeing rapid release and amplification of RNA to achieve rapid detection of RNA samples.

In a specific example, the nucleic acid release agent further includes proteinase K and lithium dodecyl sulfate, wherein proteinase K has a mass concentration ranging from 0.02 mg/mL to 1.5 mg/mL, and lithium dodecyl sulfate has a mass concentration ranging from 0.4 mg/mL to 30 mg/mL. The certain proportion of proteinase K and lithium dodecyl sulfate added may denature and degrade RNase, thereby further protecting RNA from its degradation.

In an embodiment of the present disclosure, a method for PCR amplification of a nucleic acid includes steps of: mixing the nucleic acid release agent as described above with a sample, placing the mixture at 25° C. to 60° C. for 2 min to 10 min, and adding a PCR reaction solution for PCR amplification.

The method for PCR amplification of the nucleic acid in this embodiment realizes the operation of direct amplification without extraction and purification for nucleic acid samples such as RNA, that is, the amplification and detection of the nucleic acid sample can be done by adding the nucleic acid release agent described above to the sample to release the nucleic acids from the cells, adding the PCR reaction solution and performing PCR amplification such as real-time fluorescent quantitative PCR directly, without the need for boiling and heating or extraction and purification processes, and the like. This truly realizes a one-chamber, pollution-free, simple and rapid RNA amplification and detection, with high detection sensitivity and good repeatability.

In a specific example, a volume ratio of the nucleic acid release agent to the sample is 1:1 to 1:5. Optionally, the samples can be of a variety of types including serum, plasma, an oropharyngeal swab, a nasopharyngeal swab, alveolar lavage fluid, stool and the like, and after being mixed with the nucleic acid release agent, can be directly used in detection methods downstream, such as PCR amplification or gene chip.

In a specific example, the PCR reaction solution includes deoxyribonucleoside triphosphate, a forward primer, a reverse primer, DNA polymerase, reverse transcriptase and amplification buffer, and the like. It can be understood that, according to different types and purposes of the PCR reaction, the composition of the PCR reaction solution can be selected as needed and is not limited thereto. For example, when performing real-time fluorescent quantitative PCR, the PCR reaction solution also includes fluorescent probes or fluorescent dyes, etc.

In a specific example, the method further includes a sample pretreatment step of mixing the sample with polyethylene glycol followed by centrifugation to collect a precipitate before mixing the nucleic acid release agent with the sample. Specifically, polyethylene glycol is PEG-6000, with a concentration of 0.5% to 5% by volume. Polyethylene glycol as a nucleic acid sedimentation agent can be used for the treatment of complex samples. It can effectively capture RNA viruses in free form and increase the sensitivity of later detection without affecting the PCR reaction system, and can significantly improve the performance of rapid PCR detection of RNA. For example, for cell preserving fluids containing high-salt solutions, virus preserving fluids, or hemolyzed blood samples, 50 to 5000 microliters of the sample can be taken and PEG solution in an equal volume can be added thereto followed by centrifugation at 3000 to 13000 rpm/min for 1 to 10 min, with the supernatant discarded and the pellet left, and then 50 to 100 microliters of nucleic acid release agent is added.

In a specific example, the PCR amplification of intestinal viruses in the sample is performed at following conditions:
reverse transcription at 48° C. to 52° C. for 28 min to 32 min;
thermal denaturation at 93° C. to 97° C. for 0.9 min to 1.1 min;
several cycles of amplification at 93° C. to 97° C. for 13 sec to 17 sec followed by 53° C. to 57° C. for 28 sec to 32 sec;
cooling at 23° C. to 27° C. for 8 sec to 12 sec.

In a specific example, the PCR amplification of hepatitis C virus (HCV) in the sample is performed at following conditions:
pre-denaturation and enzyme activation at 93° C. to 97° C. for 0.9 min to 1.1 min;
reverse transcription at 58° C. to 62° C. for 28 min to 32 min;
thermal denaturation at 93° C. to 97° C. for 0.9 min to 1.1 min;
several cycles of amplification at 93° C. to 97° C. for 13 sec to 17 sec followed by 58° C. to 62° C. for 28 sec to 32 sec;
cooling at 23° C. to 27° C. for 8 sec to 12 sec.

In a specific example, the PCR amplification of respiratory viruses in the sample is performed at following conditions:
reverse transcription at 48° C. to 52° C. for 28 min to 32 min;
thermal denaturation at 93° C. to 97° C. for 0.9 min to 1.1 min;
several cycles of amplification at 93° C. to 97° C. for 13 sec to 17 sec followed by 58° C. to 62° C. for 28 sec to 32 sec;
cooling at 23° C. to 27° C. for 8 sec to 12 sec.

In a specific example, the PCR amplification of respiratory bacteria in the sample is performed at following conditions:
UDG enzyme reaction at 48° C. to 52° C. for 1.9 min to 2.1 min;
thermal denaturation at 92° C. to 96° C. for 2.9 min to 3.1 min;
several cycles of amplification at 92° C. to 96° C. for 8 sec to 12 sec followed by 58° C. to 62° C. for 18 sec to 22 sec;
extension and fluorescence collection at 73° C. to 77° C. for 18 sec to 22 sec; melting curve: 62° C. to 75° C.

It can be understood that the conditions for PCR amplification are not limited to the above specific examples, and can be adjusted according to different PCR types and purposes.

In an embodiment of the present disclosure, a PCR amplification kit includes the nucleic acid release agent as described above and a PCR reaction solution. The PCR amplification kit in this embodiment realizes the operation of direct amplification without extraction and purification for nucleic acid samples such as RNA, that is, the amplification and detection of the nucleic acid sample can be done by adding the nucleic acid release agent described above to the sample to release the nucleic acids from the cells, adding the PCR reaction solution and performing PCR amplification such as real-time fluorescent quantitative PCR directly, without the need for boiling and heating or extraction and purification processes, and the like. This truly realizes a one-chamber, pollution-free, simple and rapid RNA amplification and detection, with high detection sensitivity and good repeatability.

The following are specific examples.

1. Detection for Intestinal Viruses

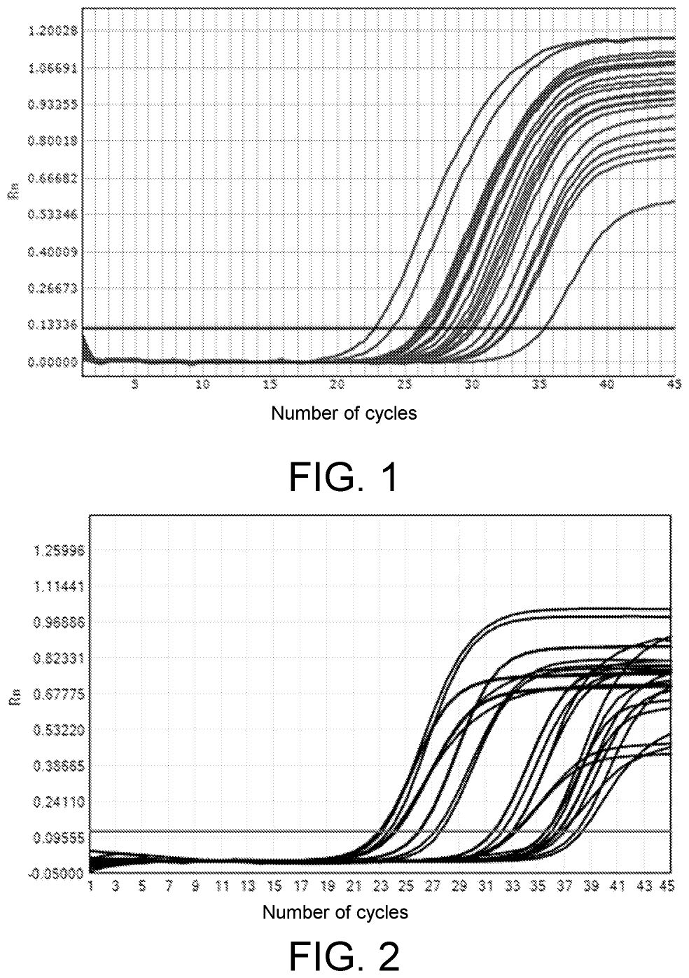

Examples 1 to 25: 25 intestinal virus throat swab samples (virus preserving fluid vehicle) were prepared. 100 μL of each sample was taken and centrifugated at 12000 rpm/min for 10 min, with the supernatant discarded. After adding 50 μL of a nucleic acid release agent and standing for 10 min, 10 μL of the nucleic acid release agent-treated sample was mixed with 40 μL of a PCR reaction solution for performing real-time fluorescence quantitative PCR amplification. The amplification curve is shown in FIG. 1. The nucleic acid release agent used in Examples 1 to 25 included Tris-HCl, sodium chloride, potassium chloride, Tween 20, Triton X-100, ethyl phenyl polyethylene glycol, betaine, bovine serum albumin, Proteinase K, lithium dodecyl sulfate and sodium hydroxide; wherein Tris-HCl had a molar concentration of 0.5 mM, sodium chloride had a molar concentration of 500 mM, Tween 20 had a volume percentage of 0.1%, Triton X-100 had a volume percentage of 3%, ethyl phenyl polyethylene glycol had a volume percentage of 0.1%, potassium chloride had a mass concentration of 8 mg/mL, sodium hydroxide had a mass concentration of 2 mg/mL, betaine had a mass concentration of 20 mg/mL, bovine serum albumin had a mass concentration of 5 mg/mL, proteinase K had a mass concentration of 1.5 mg/mL, and lithium dodecyl sulfate had a mass concentration of 0.4 mg/mL.

Comparative Examples 1-25: The above 25 samples were treated by the magnetic bead method, and real-time fluorescent quantitative PCR amplification was performed. Comparative Examples 26-35 were basically the same as Examples 1 to 10, except that Tween 20 and ethyl phenyl polyethylene glycol were not included in the nucleic acid release agent.

Comparative Examples 36-45 were basically the same as Examples 11 to 20, except that Triton X-100 was not included in the nucleic acid release agent. Comparative Examples 46-50 were basically the same as Examples 21 to 25, except that Triton X-100 in the nucleic acid release agent had a volume percentage of 8%, and potassium chloride had a mass concentration of 15 mg/mL.

Ct values in the Examples and Comparative Examples are shown in Table 1. PCR amplification was performed at following conditions:
reverse transcription at 50° C. for 30 min;
thermal denaturation at 95° C. for 1 min;
45 cycles of amplification at 95° C. for 15 sec followed by 55° C. for 30 sec;
cooling at 25° C. for 10 sec.

TABLE 1

| | Comparative | | | Comparative | |
|---|---|---|---|---|---|
| | Examples 1 | Examples | | Examples 14 | Examples |
| | to 13 | 1 to 13 | | to 25 | 14 to 25 |
| Sample 1 | 24.49 | 24.07 | Sample 14 | 20.06 | 21.68 |
| Sample 2 | 25.88 | 25.32 | Sample 15 | 24.21 | 20.64 |
| Sample 3 | 25.55 | 23.61 | Sample 16 | 23.30 | 23.63 |
| Sample 4 | 23.89 | 23.55 | Sample 17 | 26.39 | 27.63 |
| Sample 5 | 27.36 | 26.06 | Sample 18 | 29.66 | 28.03 |
| Sample 6 | 26.61 | 28.12 | Sample 19 | 24.24 | 21.44 |
| Sample 7 | 28.56 | 29.06 | Sample 20 | 34.01 | 34.23 |
| Sample 8 | 44.09 | 37.96 | Sample 21 | 28.69 | 27.57 |
| Sample 9 | 32.44 | 31.82 | Sample 22 | 27.10 | 25.31 |
| Sample 10 | 30.04 | 29.27 | Sample 23 | 29.63 | 31.11 |
| Sample 11 | 22.54 | 24.24 | Sample 24 | 31.99 | 32.50 |
| Sample 12 | 24.18 | 25.83 | Sample 25 | 27.69 | 27.60 |
| Sample 13 | 31.13 | 29.93 | | | |

According to the test results, samples that were positive for intestinal virus can be detected in all Comparative Examples 1 to 25 and Examples 1 to 25 with a consistent rate of results of 100% and good accuracy. However, the samples that were positive for intestinal virus cannot be stably and successfully detected in Comparative Examples 26-50. In addition, a smaller Ct value indicates higher detection sensitivity. Comparing the Ct values, it can be seen that in the examples using the nucleic acid release agent and the method for PCR amplification of the nucleic acid of the present disclosure, the sensitivity of the amplification and detection of RNA samples is equivalent to that using the magnetic bead method.

2. Combined Detection for Respiratory Bacteria

Examples 26 to 50: 25 respiratory tract sputum samples (normal saline vehicle) were prepared. 5 μL of each sample was taken, and 5 μL of a nucleic acid release agent was added thereto, followed by standing for 10 min. 40 μL of a PCR reaction solution was added with mixing for performing real-time fluorescence quantitative PCR amplification. The amplification curve is shown in FIG. 2. The nucleic acid release agents used included Tris-HCl, sodium chloride, potassium chloride, Tween 20, Triton X-100, ethyl phenyl polyethylene glycol, betaine, bovine serum albumin, proteinase K, lithium dodecyl sulfate and sodium hydroxide; wherein Tris-HCl had a molar concentration of 500 mM, sodium chloride had a molar concentration of 20 mM, Tween 20 had a volume percentage of 2%, Triton X-100 had a volume percentage of 0.1%, ethyl phenyl polyethylene glycol had a volume percentage of 3%, potassium chloride had a mass concentration of 5 mg/mL, sodium hydroxide had a mass concentration of 50 mg/mL, betaine had a mass concentration of 0.1 mg/mL, bovine serum albumin had a mass concentration of 100 mg/mL, proteinase K had a mass concentration of 0.02 mg/mL, and lithium dodecyl sulfate had a mass concentration of 30 mg/mL.

Comparative Examples 51-75: The above 25 samples were treated by the magnetic bead method, and real-time fluorescent quantitative PCR amplification was performed.

Ct values in the Examples and Comparative Examples are shown in Table 2. PCR amplification was performed at following conditions:
UDG enzyme reaction at 50° C. for 2 min;
thermal denaturation at 94° C. for 3 min;
45 cycles of amplification at 94° C. for 10 sec followed by 60° C. for 20 sec;
extension and fluorescence collection at 75° C. for 20 sec;
melting curve: 62° C. to 75° C.

TABLE 2

| | Comparative | | | Comparative | |
|---|---|---|---|---|---|
| | Examples 51 | Examples | | Examples 64 | Examples |
| | to 63 | 26 to 38 | | to 75 | 39 to 50 |
| Sample 1 | 35.36 | 31.34 | Sample 14 | 23.35 | 24.14 |
| Sample 2 | 29.47 | 24.54 | Sample 15 | 36.03 | 32.69 |
| Sample 3 | 30.18 | 31.1 | Sample 16 | 36.2 | 35.99 |
| Sample 4 | 37.23 | 36.56 | Sample 17 | 36.02 | 35.95 |
| Sample 5 | 33.21 | 28.44 | Sample 18 | 33.44 | 33.2 |
| Sample 6 | 27.94 | 25.69 | Sample 19 | 38.34 | 38.32 |
| Sample 7 | 26.58 | 24.945 | Sample 20 | 30.76 | 29.145 |
| Sample 8 | 31.19 | 30.495 | Sample 21 | 36.49 | 30.85 |
| Sample 9 | 33.64 | 29.675 | Sample 22 | 26.22 | 25.925 |
| Sample 10 | 37.95 | 34.455 | Sample 23 | 23.01 | 22.96 |
| Sample 11 | 32.68 | 24.13 | Sample 24 | 31.39 | 27.32 |
| Sample 12 | 27.58 | 23.355 | Sample 25 | 29.26 | 24.015 |
| Sample 13 | 34.14 | 32.605 | | | |

According to the test results, samples that were positive for respiratory bacteria can be detected in all comparative examples and examples with a consistent rate of results of 100% and good accuracy. In addition, a smaller Ct value indicates higher detection sensitivity. Comparing the Ct values, it can be seen that in the examples using the nucleic acid release agent and the method for PCR amplification of the nucleic acid of the present disclosure, the sensitivity of the multiple amplification and detection of bacteria was equivalent to that using the magnetic bead method. It also shows that the nucleic acid release agent and the method for PCR amplification of the nucleic acid of the present disclosure are not only applicable to amplification and detection of RNA samples, but also can be applied to amplification and detection of DNA samples.

3. Detection for HCV Viruses

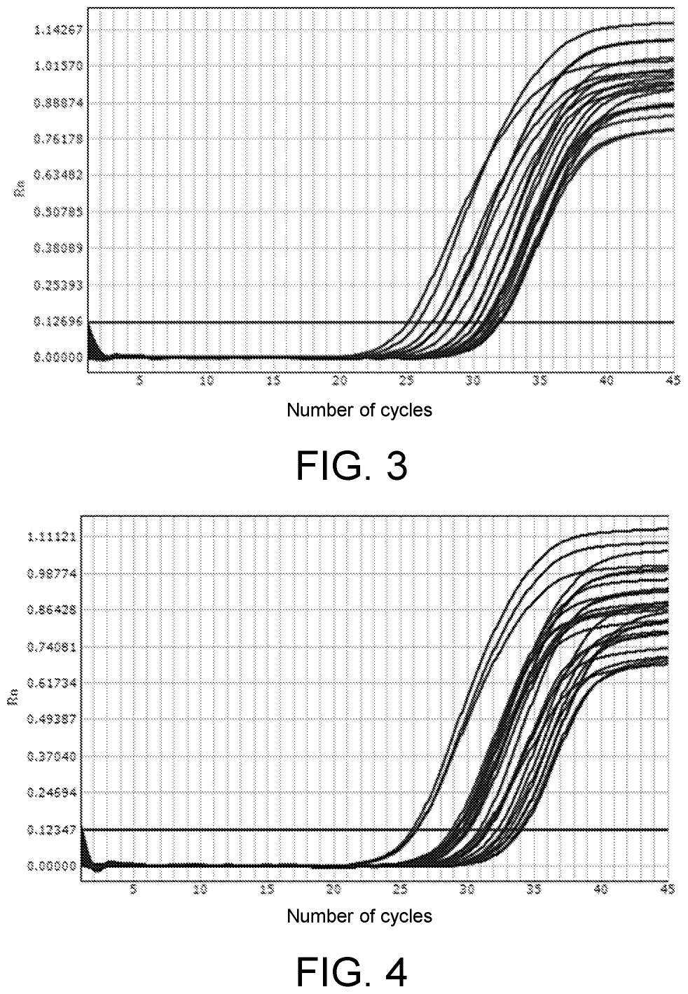

Examples 51 to 70: 20 HCV serum samples were prepared. 15 μL of each sample was taken, and 5 μL of a nucleic acid release agent was added thereto, followed by standing for 10 min. 30 μL of a PCR reaction solution was added with mixing for performing real-time fluorescence quantitative PCR amplification. The amplification curve is shown in FIG. 3. The nucleic acid release agent used included Tris-HCl, sodium chloride, potassium chloride, Tween 20, Triton X-100, ethyl phenyl polyethylene glycol, betaine, bovine serum albumin, and sodium hydroxide; wherein Tris-HCl had a molar concentration of 200 mM, sodium chloride had a molar concentration of 250 mM, Tween 20 had a volume percentage of 1%, Triton X-100 had a volume percentage of 2%, ethyl phenyl polyethylene glycol had a volume percentage of 2%, potassium chloride had a mass concentration of 7 mg/mL, sodium hydroxide had a mass concentration of 25 mg/mL, betaine had a mass concentration of 10 mg/mL, and bovine serum albumin had a mass concentration of 60 mg/mL.

Examples 91 to 110: the same HCV serum samples as in Examples 51 to 70 were used. 15 μL of each sample was taken, and 5 μL of a nucleic acid release agent was added thereto, followed by standing for 10 min. 30 μL of a PCR reaction solution was added with mixing for performing real-time fluorescence quantitative PCR amplification. The nucleic acid release agent is the same as the nucleic acid release agent used in Examples 1 to 25.

Ct values in the examples are shown in Table 3. PCR amplification was performed at following conditions:
pre-denaturation and enzyme activation at 95° C. for 1 min;
reverse transcription at 60° C. for 30 min;
thermal denaturation at 95° C. for 1 min;
45 cycles of amplification at 95° C. for 15 sec followed by 60° C. for 30 sec;
cooling at 25° C. for 10 sec.

TABLE 3

| | Examples | Examples | | Examples | Examples |
|---|---|---|---|---|---|
| | 91 to 100 | 51 to 60 | | 101 to 110 | 61 to 70 |
| Sample 1 | 30.25 | 31.32 | Sample 11 | 24.56 | 25.13 |
| Sample 2 | 27.66 | 29.78 | Sample 12 | 25.60 | 26.03 |
| Sample 3 | 28.31 | 30.28 | Sample 13 | 27.06 | 27.92 |
| Sample 4 | 29.00 | 31.23 | Sample 14 | 27.95 | 28.88 |
| Sample 5 | 28.63 | 31.81 | Sample 15 | 31.05 | 31.64 |
| Sample 6 | 27.25 | 27.94 | Sample 16 | 28.60 | 30.55 |
| Sample 7 | 27.21 | 26.85 | Sample 17 | 29.67 | 30.76 |
| Sample 8 | 29.29 | 31.27 | Sample 18 | 30.44 | 31.49 |
| Sample 9 | 28.63 | 31.64 | Sample 19 | 19.78 | 20.25 |
| Sample 10 | 30.55 | 31.95 | Sample 20 | 33.20 | 32.01 |

According to FIG. 3, it can be seen that in samples that were positive for HCV can be detected in all examples using the nucleic acid release agent and the method for PCR amplification of the nucleic acid of the present disclosure with good accuracy. In addition, it can be seen from Table 3 that the detection sensitivity in Examples 51 to 70 is slightly worse than that in Examples 91 to 100, indicating that the effect of the nucleic acid release agent used in Examples 51 to 70 is slightly inferior to that of the nucleic acid release agent used in Examples 91 to 100.

4. Detection for Respiratory Viruses

Examples 71 to 90: 20 respiratory virus throat swab samples (normal saline vehicle) were prepared. 100 μL of each sample was taken and centrifugated at 12000 rpm/min for 10 min, with the supernatant discarded. After adding 50 μL of a nucleic acid release agent and standing for 10 min, 10 μL of the nucleic acid release agent-treated sample was mixed with 40 μL of a PCR reaction solution for performing real-time fluorescence quantitative PCR amplification. The amplification curve is shown in FIG. 1. The nucleic acid release agent used included Tris-HCl, sodium chloride, potassium chloride, Tween 20, Triton X-100, ethyl phenyl polyethylene glycol, and sodium hydroxide; wherein Tris-HCl had a molar concentration of 400 mM, sodium chloride had a molar concentration of 150 mM, Tween 20 had a volume percentage of 0.8%, Triton X-100 had a volume percentage of 1.2%, ethyl phenyl polyethylene glycol had a volume percentage of 1.5%, potassium chloride had a mass concentration of 6 mg/mL, and sodium hydroxide had a mass concentration of 15 mg/mL.

Examples 111 to 130: the same respiratory virus throat swab samples as in Examples 71 to 90 were used. 100 μL of each sample was taken and centrifugated at 12000 rpm/min for 10 min, with the supernatant discarded. After adding 50 μL of a nucleic acid release agent and standing for 10 min, 10 μL of the nucleic acid release agent-treated sample was mixed with 40 μL of a PCR reaction solution for performing real-time fluorescence quantitative PCR amplification. The nucleic acid release agent was the same as the nucleic acid release agent used in Examples 51 to 70.

Ct values in the examples are shown in Table 4. PCR amplification was performed at following conditions:
reverse transcription at 50° C. for 30 min;
thermal denaturation at 95° C. for 1 min;
45 cycles of amplification at 95° C. for 15 sec followed by 60° C. for 30 sec;
cooling at 25° C. for 10 sec.

TABLE 4

| | Examples | Examples | | Examples | Examples |
|---|---|---|---|---|---|
| | 111 to 120 | 71 to 80 | | 121 to 130 | 81 to 90 |
| Sample 1 | 23.64 | 26.05 | Sample 11 | 27.56 | 28.69 |
| Sample 2 | 25.97 | 28.77 | Sample 12 | 28.67 | 29.61 |
| Sample 3 | 29.55 | 31.28 | Sample 13 | 26.22 | 28.32 |
| Sample 4 | 30.52 | 32.65 | Sample 14 | 27.88 | 29.78 |
| Sample 5 | 28.10 | 28.55 | Sample 15 | 31.36 | 31.54 |
| Sample 6 | 31.69 | 34.12 | Sample 16 | 30.69 | 33.69 |
| Sample 7 | 25.19 | 26.12 | Sample 17 | 29.89 | 29.47 |
| Sample 8 | 30.26 | 32.36 | Sample 18 | 30.79 | 32.34 |
| Sample 9 | 28.96 | 31.59 | Sample 19 | 29.20 | 32.69 |
| Sample 10 | 31.60 | 31.35 | Sample 20 | 33.59 | 33.06 |

According to FIG. 4, it can be seen that samples that were positive for respiratory virus can be detected in all examples using the nucleic acid release agent and the method for PCR amplification of the nucleic acid of the present disclosure with good accuracy. In addition, it can be seen from Table 4 that the detection sensitivity in Examples 71 to 90 was slightly worse than that in Examples 111 to 130, indicating that the effect of the nucleic acid release agent used in Examples 71 to 90 was slightly inferior to that of the nucleic acid release agent used in Examples 111 to 130.

The technical features of the above-described embodiments may be combined arbitrarily. To simplify the description, not all of the possible combinations of the technical features in the above embodiments are described. However, all of the combinations of these technical features should be considered as within the scope of the present disclosure, as long as such combinations do not contradict with each other.

The above-described embodiments merely represent several embodiments of the present disclosure, and the description thereof is more specific and detailed, but it should not be construed as limiting the scope of the present disclosure. It should be noted that, for those skilled in the art, several variations and improvements may be made without departing from the concept of the present disclosure, and these are all within the protection scope of the present disclosure. Therefore, the scope of the present disclosure shall be defined by the appended claims.

1. A nucleic acid release agent, comprising Tris-HCl, sodium chloride, potassium chloride, Tween 20, Triton X-100, ethyl phenyl polyethylene glycol and a strong base; wherein the Tris-HCl has a molar concentration ranging from 0.5 mM to 500 mM, the sodium chloride has a molar concentration from 20 mM to 500 mM, the potassium chloride has a mass concentration ranging from 5 mg/mL to 8 mg/mL, the Tween 20 has a volume percentage ranging from 0.1% to 2%, the Triton X-100 has a volume percentage ranging from 0.1% to 3%, the ethyl phenyl polyethylene glycol has a volume percentage ranging from 0.1% to 3%, and the strong base has a mass concentration ranging from 2 mg/mL to 50 mg/mL.

2. The nucleic acid release agent according to claim 1, further comprising betaine and bovine serum albumin, wherein the betaine has a mass concentration ranging from 0.1 mg/mL to 20 mg/mL, and the bovine serum albumin has a mass concentration ranging from 5 mg/mL to 100 mg/mL.

3. The nucleic acid release agent according to claim 1, further comprising proteinase K and lithium dodecyl sulfate, wherein the proteinase K has a mass concentration ranging from 0.02 mg/mL to 1.5 mg/mL, and the lithium dodecyl sulfate has a mass concentration ranging from 0.4 mg/mL to 30 mg/mL.

4. A method for PCR amplification of a nucleic acid, comprising the following steps of: mixing the nucleic acid release agent according to claim 1 with a sample, placing the mixture at 25° C. to 60° C. for 2 min to 10 min, and adding a PCR reaction solution for PCR amplification.

5. The method according to claim 4, further comprising a sample pretreatment step of: mixing the sample with polyethylene glycol followed by centrifugation to collect a precipitate before mixing the nucleic acid release agent with the sample.

6. The method according to claim 4, wherein the PCR amplification of intestinal viruses in the sample is performed at following conditions:
reverse transcription at 48° C. to 52° C. for 28 min to 32 min;
thermal denaturation at 93° C. to 97° C. for 0.9 min to 1.1 min;
several cycles of amplification at 93° C. to 97° C. for 13 sec to 17 sec followed by 53° C. to 57° C. for 28 sec to 32 sec;
cooling at 23° C. to 27° C. for 8 sec to 12 sec.

7. The method according to claim 4, wherein the PCR amplification of hepatitis C virus in the sample is performed at following conditions:
pre-denaturation and enzyme activation at 93° C. to 97° C. for 0.9 min to 1.1 min;
reverse transcription at 58° C. to 62° C. for 28 min to 32 min;
thermal denaturation at 93° C. to 97° C. for 0.9 min to 1.1 min;
several cycles of amplification at 93° C. to 97° C. for 13 sec to 17 sec followed by 58° C. to 62° C. for 28 sec to 32 sec;
cooling at 23° C. to 27° C. for 8 sec to 12 sec.

8. The method according to claim 4, wherein the PCR amplification of respiratory viruses in the sample is performed at following conditions:
reverse transcription at 48° C. to 52° C. for 28 min to 32 min;
thermal denaturation at 93° C. to 97° C. for 0.9 min to 1.1 min;
several cycles of amplification at 93° C. to 97° C. for 13 sec to 17 sec followed by 58° C. to 62° C. for 28 sec to 32 sec;
cooling at 23° C. to 27° C. for 8 sec to 12 sec.

9. The method according to claim 4, wherein the PCR amplification of respiratory bacteria in the sample is performed at following conditions:
UDG enzyme reaction at 48° C. to 52° C. for 1.9 min to 2.1 min;
thermal denaturation at 92° C. to 96° C. for 2.9 min to 3.1 min;
several cycles of amplification at 92° C. to 96° C. for 8 sec to 12 sec followed by 58° C. to 62° C. for 18 sec to 22 sec;
extension and fluorescence collection at 73° C. to 77° C. for 18 sec to 22 sec;
melting curve: 62° C. to 75° C.

10. A PCR amplification kit, by comprising the nucleic acid release agent according to claim 1 and a PCR reaction solution.
